(19) United States
(12) Patent Application Publication
(10) Pub. No.: US 2019/0015047 A1
(43) Pub. Date: Jan. 17, 2019

(54) A METHOD AND APPARATUS FOR EUSTACHIAN TUBE DYSFUNCTION ASSESSMENT

(71) Applicant: Tarik Ozkul, Sharjah (AE)

(72) Inventors: Tarik Ozkul, Sharjah (AE); Murat Haluk Ozkul, Istanbul (TR)

(73) Assignee: Tarik Ozkul, Sharjah (AE)

(21) Appl. No.: 16/076,821

(22) PCT Filed: Feb. 8, 2017
(86) PCT No.: PCT/IB2017/050680

§ 371 Date: Aug. 9, 2018

Related U.S. Application Data
(63) Continuation of application No. 15/018,874, filed on Feb. 9, 2016, now abandoned.

(30) Foreign Application Priority Data
Mar. 17, 2016 (TR) 2016/03458

(51) Int. Cl. A61B 5/00; A61B 5/12; A61N 1/05; A61B 5/03

(52) U.S. Cl. CPC A61B 5/6819; A61B 5/12; A61B 5/682; A61N 1/36036; A61N 1/0546; A61B 5/038; A61N 1/0504

(57) ABSTRACT

This invention is about an apparatus and method for positive identification of patients suffering from Eustachian Tube Dysfunction ailment. Eustachian Tube dysfunction is a common ailment which almost 1% of world population suffers. Recent clinical study indicated that some of the Eustachian Tube Dysfunction cases are due to lack of synchronization and synergy of the two muscles responsible for opening Eustachian Tube. The invention teaches a device and method for assessment of Eustachian Tube Dysfunction and identifies patients where dysfunction problem originate from lack of muscle synchronization.

### FIELD

This invention is related to a method and device used for measuring Eustachian dysfunction level of patients.

### BACKGROUND OF THE INVENTION

The Eustachian tube is a hollow tube that originates in the back of the nose and connects the nasal cavity to middle ear space. The middle ear space is the hollowed out portion of the skull bone that contains the hearing mechanism, which is covered by eardrum on one side and cochlea on the other side. The Eustachian Tube provides ventilation, drainage and protection of middle ear space against reflux, microorganisms, excessive sound pressure and pressure changes in pharynx. In adults, the Eustachian tube is approximately 35 mm long and approximately 3 mm in diameter. The first part of the Eustachian tube is supported by cartilage and the last part that is close to the middle ear space is located inside the bone structure. The lining tissue of the Eustachian tube is similar to the tissue that covers the nasal cavity and responds to external stimulants just as nasal tissue does.

The main function of the Eustachian tube is to provide ventilation to the middle ear space, ensuring that its pressure remains at slightly negative but close to ambient air pressure. Another function of the Eustachian tube is to drain secretions and debris from the middle ear space. Several small muscles located in the back of the throat and palate control the opening and closing action of the tube. Typical muscular actions like swallowing and yawning cause contractions of these muscles and activate Eustachian tube function time to time to achieve its pressure regulation function.

Eustachian tube is normally closed most of the time to prevent contaminants contained in the nasal cavity to reach the middle ear cavity. Disorders of Eustachian Tube are either “dilatory” type (failure to open adequately) or “patulous” type (failure to maintain tube closed at rest). If dysfunction of Eustachian tube makes the tube always open, this condition is called “patulous” type dysfunction of the Eustachian tube. Patients with “patulous” dysfunctional Eustachian tube suffer from frequent, chronic ear infections. A more common form of the Eustachian tube dysfunction is caused by partial or complete blockage of the Eustachian tube, which causes sensations of popping, clicking, and ear fullness as well as moderate to severe ear pain associated with the condition. This type of dysfunction is the “dilatory” type dysfunction of the Eustachian Tube.

Eustachian tube dysfunction (ETD) is medically defined as “the inability of the Eustachian tube to perform its required functions”, e.g. opening up regularly to pressure equalize the middle ear cavity. A recently published book titled “Interventions for adult Eustachian tube dysfunction: a systematic review” by Llewellyn A, Norman G, Harden M, et al. Health Technology Assessment, No. 18.46, Southampton (UK): MEM Journals Library, July 2014 can be considered as an authoritative book summarizing current practices and technologies used in Eustachian tube function related matters. Excerpts from this reference book will be used for establishing facts related to Eustachian tube assessment.

Diagnosis of Eustachian tube dysfunction (ETD) is not easy. According to the reference book mentioned above;
“There are no comprehensive guidelines on diagnosis of ETD. Diagnosis is generally based on medical history and clinical examination to identify potential underlying causes. Currently, there is no commonly used patient-reported outcome measure. A scale for the assessment of ETD [the 7-item Eustachian Tube Dysfunction Questionnaire (ETDQ-7)] was tested for validity; this is a questionnaire addressing a range of symptoms associated with ETD, which is completed by the patient . . . The lack of clearly defined diagnostic criteria, together with the uncertainty relating to the etiology of ETD, presents a key challenge in undertaking a review of interventions for its treatment. Lack of consensus on the necessary features for diagnosis, including clinical history, requires additional awareness of the risk of error and bias in the selection of studies, as well as increasing the probability of clinical heterogeneity in the included studies.” End of quote.

As it is mentioned above, there is no consensus on diagnosis or assessment of ETD. Most common practice used in identifying ETD is through tympanometry measurement. According to the reference mentioned above; “The UK national survey defined ETD as the presence of a normal or abnormal but intact tympanic membrane with a middle ear pressure of <−100 mmH2O and an air-bone gap of ≥15 decibels (dB)”. Tympanometry tests can only be performed on patients with healthy tympanic membrane. Since most chronic ETD patients have ruptured tympanic membrane due to their condition, tympanometry cannot be used on such patients.

Recent clinical study done by inventors revealed an interesting finding about ETD. The study revealed that individuals with healthy Eustachian tubes receive periodic neural signals to their Eustachian tube muscles. The signals are highly periodic and come with a specific pattern and phase difference between them. Clinical study revealed that patients with ETD problem have indeed problems with neural signals received by the Eustachian tube muscles. The problem appears either with the periodicity of the signals, or the latency between the signals or the amplitude of the signals. Based on the guidelines of this clinical study, a new type of ETD assessment device is designed which assesses the ETD level of the patients according to the signal pattern and functionality of ET muscles.

### SUMMARY

The invention presented in this document aims to assess condition of ET dysfunction in patients regardless of the condition of their tympanic membrane. It further aims to identify patients who have ET dysfunction due to neurological disorder of the muscles controlling Eustachian Tube.

The basis of the invention starts from a discovery made by inventors during a clinical investigation of the Eustachian Tube muscle behavior. Medical community so far believed that Eustachian tube opens sporadically only during yawning or swallowing action. During clinical trials inventors noticed that Eustachian tube muscles receive neurological signals periodically almost every 20 seconds and activate periodically. The periodicity of the signals resembles almost a heartbeat signal albeit slower. Due to intricate anatomy of the Eustachian tube valve, the muscles need to work synergistically in order to open the valve. One of the muscles needs to be activated first, followed by the other muscle after a precise delay in order to open the Eustachian tube. Any discoordination of the muscle activity causes muscles to contradict to each other, which in turn results in dysfunctional Eustachian tube. After studying dozens of patients, it appeared that the cause of Eustachian Dysfunction in some patients is due to lack of synchronization of neural signals received by the ET muscles. It also appeared that by applying a certain signal pattern to Eustachian Tube muscles through the palate of the patient by subdermal electrodes, it is possible to open the Eustachian tube. Most researchers and medical practitioners so far tried to access Eustachian tube muscles nasally through nose. Nasal area is very sensitive and application of subdermal electrodes in this area is quite painful for most patients. So most researchers use anesthetics to alleviate the pain while recoding muscle activity. Since anesthetics interfere with nerve activity, real nature of the signals so far has never been revealed. Inventors are the first researchers who accessed ET muscles through the palate without using anesthetics and recorded the signals. Analyses of the signals revealed important synergistic and periodic nature of the neural signals.

Eustachian tube is not a simple tube, but a delicate valve controlled by two muscles. These two tubal muscles are the Levator Veli Palatini Muscle (mLVP) and the Tensor Veli Palatini muscle (mTVP). Among the two, Tensor Veli Palatini (mTVP) is the main tubal dilator which performs the Eustachian tube function. However, coordinated synergistic action of mLVP and mTVP together performs an efficient opening action of Eustachian Tube. Any disturbance of the synergy between these two muscles may make them work antagonist to each other which result in ET dysfunction.

Making use of these discoveries, the newly invented diagnostic device tests Eustachian Tube and assesses ETD condition of the patient by doing the following tests. Test steps may be executed fully or partially to assess the ETDA (Eustachian Tube Dysfunction Assessment) condition of the patient.

mLVP and mTVP muscles of the patient are stimulated electrically or optically by using probes placed to the palate of the patient while monitoring the opening of Eustachian tube through sensors placed into external ear canal. In case of electrical stimulation, the probes are subdermal electrodes, in case of optical stimulation they are fiber optic cables or light emitting diode (led) placed at the tip of the probe. While administering subdermal electrodes the patient is not anesthetized, however does not feel any pain at the particular application location due to insensitive nature of the application area and miniscule size of subdermal electrodes. An alternative way of stimulating ET muscles is through optical stimulation. Optical stimulation is a new technique for stimulating muscles and very suitable for this particular application. Optical stimulation pulses are applied through fiber optic cables or by photodiodes installed on distal end of a probe assembly. In both cases optical energy is directed to the same location transpalatally where subdermal electrodes were applied.

Eustachian tube dysfunction assessment is done as follows:
1. A steady pressure maintained inside the nasal cavity of the patient during the test makes it easier to observe Eustachian opening through external ear canal sensor. Maintaining pressure inside the nasal cavity is done using an external pressure generator pumping air or instructing patient apply pressure himself/herself as it is done in Valsalva maneuver. (Forcing air into nasal cavity.) During stimulation session stimulations are applied in an organized pattern. First, mTVP muscle is stimulated only and Eustachian tube opening is observed by using the sensors placed into the external ear canal. If ET opening not successful, mLVP muscle is stimulated and Eustachian tube opening is observed. If ET opening is still not successful, both mLVP and mTVP muscles are stimulated together while mLVP is stimulated first, followed by mTVP with 60-300 millisecond latency.
2. If Eustachian tube does not open in step 1, step 1 process is repeated with changed parameters. Parameters are changed by increasing signal amplitude of stimulation, increasing the latency between the stimulation signals given to mLVP, mTVP muscles and then observing the Eustachian tube opening again.

As a result of these tests, it will be possible to identify if the patient has ETD condition due to neurological disorder or due to some other reason. The invention provides clinical evidence about the condition of the ET muscles for the health professional to decide. Having Eustachian tube open with only mTVP stimulation, or only with mLVP stimulation, or with both mTVP and mLVP stimulation together tell information about the state of Eustachian tube dysfunction. Those patients whose ET opens due to stimulation of mTVP, mLVP are a category of patients who are likely to be treated by correcting this neurological disorder. There is a branch of medical practice called “neuromodulation therapy” which aims to correct this sort of disorders. The purpose of the invention is to assess the ETD condition of patients and identify those who may get benefit from neurological treatment.

### BRIEF DESCRIPTION OF DRAWINGS

FIG. 1 shows location of Eustachian tube muscles mTVP and mLVP as it is seen from the mouth of the patient. This the location where the signals are picked up and stimulation is applied;

FIG. 2A shows the neurological signals mLVP and mTVP of a subject with healthy Eustachian tube activity;

FIG. 2B shows the neurological signals mLVP and mTVP of a subject with dysfunctional Eustachian tube;

FIG. 3 shows functional block diagram of the Eustachian tube dysfunction assessment device;

FIG. 4A shows a patient during assessment test with sensors and electrical stimulation leads connected;

FIG. 4B shows a patient during assessment test with sensors and optical stimulation leads connected;

FIG. 5 shows the software flow chart of the operation of the device;

FIG. 6 shows shape of signals applied to mTVP, mLVP to stimulate the muscles;

FIG. 7 shows the output of the sensor reading indicating Eustachian tube activity when muscles of Eustachian tube are stimulated electrically or optically.

### DESCRIPTION

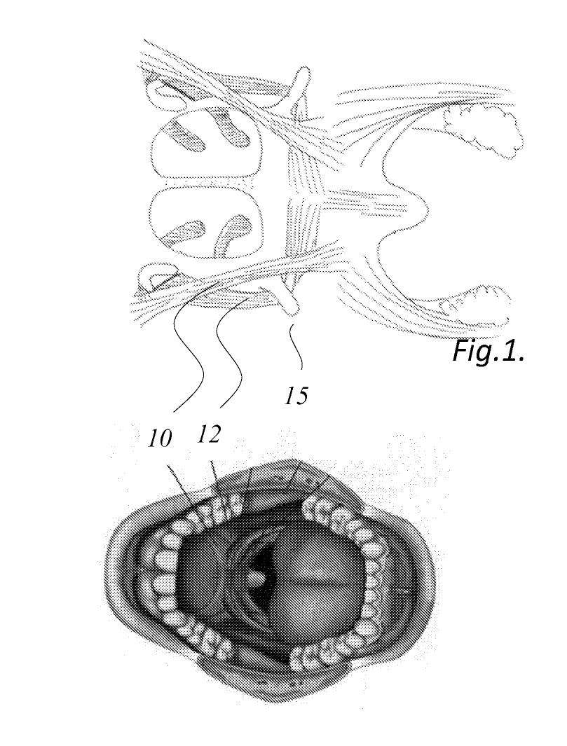

The operation of the present invention will now be described with the aid of the figures. The purpose of the invention is to diagnose the Eustachian tube dysfunction cases and identify the ones that are suffering because of neurological disorder of Eustachian tube (ET) muscles. As it is explained in the background section of this document, so far Eustachian tube dysfunction (ETD) ailment has been an ill-defined condition with little knowledge about the underlying cause. The invention is intended to diagnose ETD condition in patients and identify the category of patients who have healthy ET muscles and ET valve mechanism, but suffering from ETD because of disorder of neurological signals received by the muscles. Identification of these patients is important because these patients may benefit from neurological therapy. The invention does this by stimulating Eustachian tube muscles of the patients while monitoring pressure in the external ear canal. Stimulation is done from an easily accessible and identifiable location without causing excessive pain or discomfort to patients. FIG. 1 shows electromyography (EMG) signal pick up locations for Levator Veli Palatini muscle (mLVP) 10 and Tensor Veli Palatini muscle (mTVP) 12. Number 15 shows the location of Hamulus Pterygoids which is not apparent visibly inside the mouth of the patient, but palpable by experienced physicians. Identification of location of Hamulus Pterygoids is important for accurately locating mLVP and mTVP muscles.

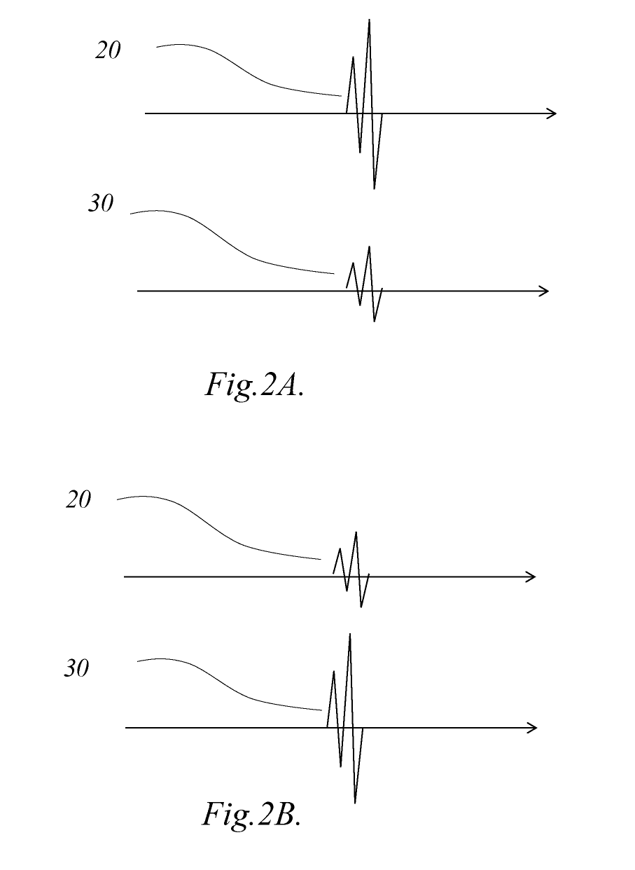

The amplitude, periodicity and phase difference between the two signals received from locations 10 and 12 tell important information about the health of Eustachian tube activity. FIG. 2A shows activity of healthy Eustachian tube signals, mLVP signal (20) and mTVP signal (30) as they are observed on a computer screen. In healthy subjects with no ETD symptoms, signal 20 is observed to be bigger in signal amplitude than signal 30, and signal 20 always starts before signal 30. FIG. 2B. shows mLVP and mTVP signals for an ETD case where mTVP signal 30 is bigger in signal amplitude than mLVP signal 20 and signal 30 starts before signal 20. This is a neurological disorder. Clinical trials indicated that at least some of the ETD cases is related to this type of neurological disorder. Clinical study indicated that when this type of patients are stimulated by a 0.02 msec. duration external pulses with 0.2 mV amplitude, the Eustachian tube of the patient opens. This is an indication that this type of patients have intact muscular system and likely to respond to neuromodulation therapy for curing the ailment. It is the objective of this invention to diagnose and identify this kind of patients.

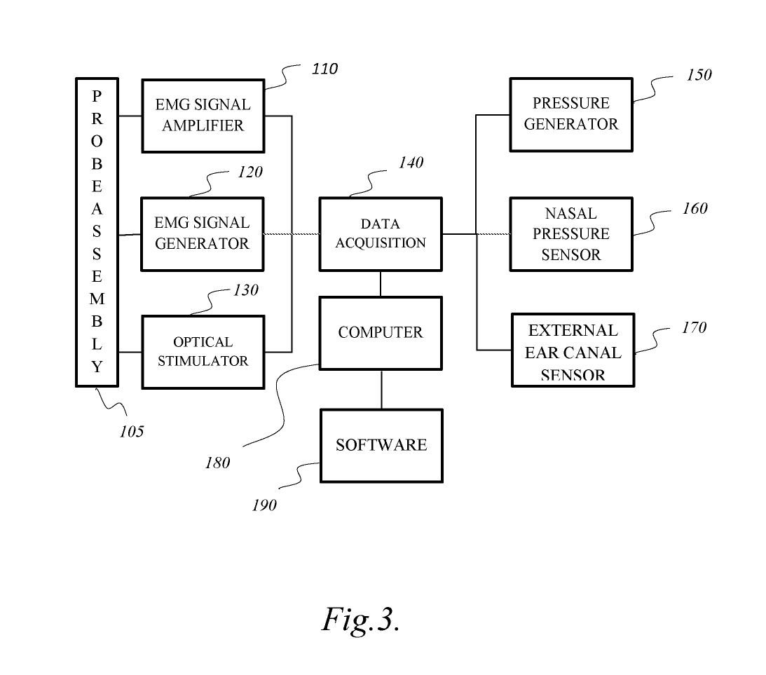

FIG. 3 shows the main components of the invention. EMG signals mLVP and mTVP are received from the patient and connected to block 110 through probe assembly 105 which receive and amplify the mLVP, mTVP EMG signals. Block 110 is an incoming signal conditioner that amplifies mLVP, mTVP signals. Block 120 is an outgoing signal conditioner used for generating external stimulation pulses to open the Eustachian tube. Block 130 is another outgoing signal conditioner that generates optical pulses to stimulate the muscles. The blocks 110, 120 and 130 are connected to data acquisition block 140 which converts received signals into digital form and give it to block 180 which is the Computer. Block 190 is software and controls the actions of the instrument. Block 150 is a pressure generator which provides low pressure air required for the testing process. Block 160 is a nasal pressure sensor which measures the pressure inside nasal cavity. Block 170 is external ear canal sensor placed in the ear of the patient.

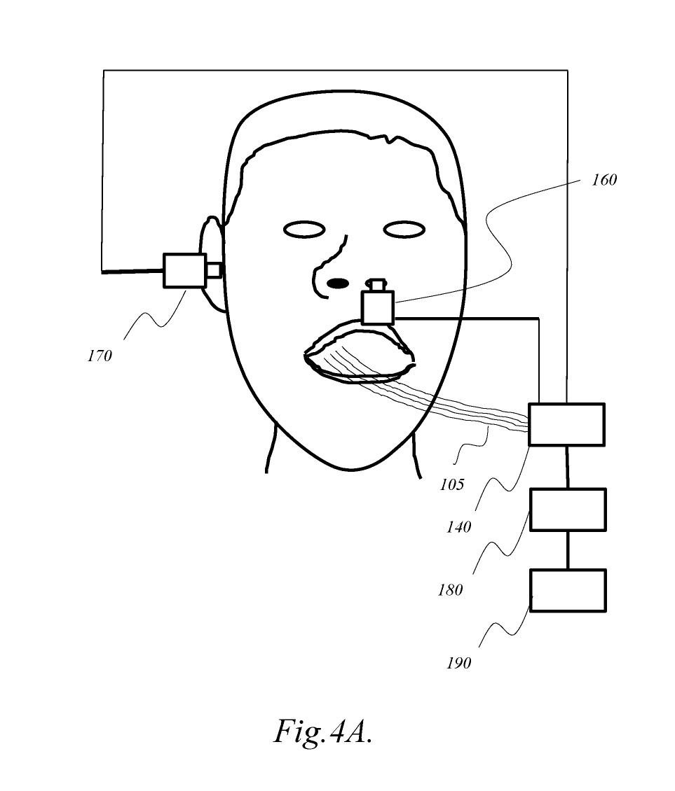

FIG. 4A shows the connection of the EMG electrodes and sensors to the patient during a typical diagnostic session. Number 105 shows probe assembly with mLVP and mTVP subdermal electrodes together with the reference electrodes. Number 170 is the external ear canal sensor and number 160 is the nasal pressure sensor. 140 is the data acquisition system where the probe assembly 105 is connected via signal conditioner blocks. Computer 180 and the software 190 together controls the stimulation pulses and monitors external ear canal sensor 170 and nasal pressure sensor 160 to assess the dysfunction level.

FIG. 4B shows another embodiment of the invention where optical stimulation is used for stimulating mTVP, mLVP muscles instead of subdermal EMG stimulation electrodes. Optical nerve stimulation is an alternative way of stimulating muscles and it is well known by specialists. It is especially useful for patients with needle phobia who have needle anxiety. Number 135 indicates optical stimulation probes used for mLVP and mTVP stimulation. The figure also shows an alternative way of measuring pressure through mask 165 which covers both nose and mouth of the patient. Nasal pressure sensor 160 is installed on the mask 165. Yet another alternative method is placing the pressure sensor 160 in the mouth of the patient while patient keeps the mouth closed.

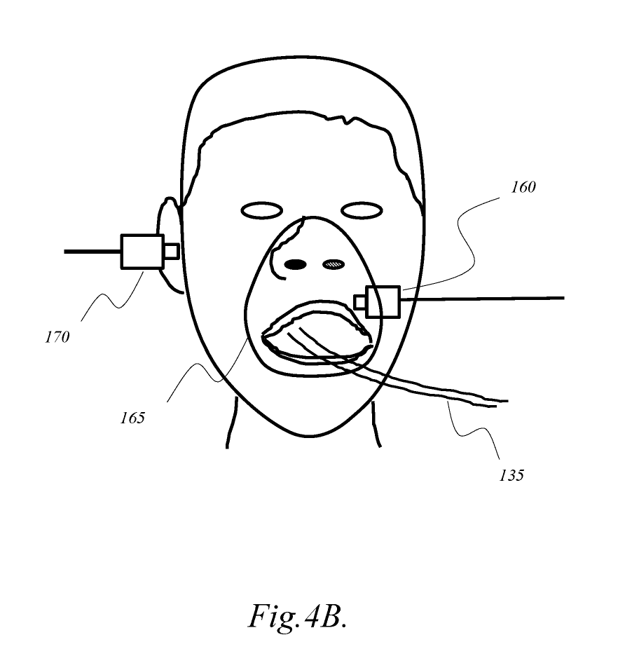

FIG. 5 shows flowchart of the operation of the invention. Diagnosis and assessment operation starts by computer reading electrical signals coming from mLVP and mTVP electrodes while patient is at rest. Block 210 shows where reading and recoding the mLVP, mTVP signals is done for minimum of 150 seconds. The reading and recoding activity can extend up to 300 seconds. As a next step, the recorded signals are displayed on the computer screen in block 220.

In another embodiment of the system reading signals (210), and displaying signals (220) may be omitted and operation of the system may start from block 230 directly.

In the next phase of the assessment, pressure generator is started (230) and maximum of 50 daPa pressure is applied to nasal cavity of the patient through nasal pressure sensor pathway. Pressurization of nasal cavity continues until pressure reaches 50 daPa (240). 50 daPa pressure level is considered “mild” pressure level which is tolerable by most patients. In another embodiment of the system, the pressure level may be adjustable for those patients who may find 50 daPa uncomfortably high.

As a next step, the stimulation pulses are applied to the patient in block 250. Stimulation is given either electrically or optically to mLVP and mTVP muscles. As a first step, only mTVP muscle is stimulated with electrical signal of 0.2 mV amplitude pulse with 0.020 msec. duration. During the application of the pulse, external ear canal sensor output is monitored. Opening of ET is seen as a distinct change in the output of external ear canal sensor output with at least 10 daPa pressure change (270). If the pressure change in not observed, the stimulation step is repeated after changing parameters (280). During this step, both mLVP and mTVP are stimulated by 0.2 mV pulses while mLVP receives longer duration pulse which lasts 0.040 msec. while mTVP receives 0.020 msec. duration pulse. The mLVP muscle stimulation pulse is applied 0.020 msec. before the mTVP pulse is applied. During the stimulation, the external ear canal sensor is monitored again for at least 10 daPa pressure change. If no pressure change is observed, the signal amplitude is increased to 0.3 mV and stimulation is repeated. Although these are the recommended parameters based on the clinical trials, in another embodiment of the invention, the pulse amplitude and pulse duration is made adjustable for finding the best pulse pattern for the patients. Block 260 shows the step where the test results are displayed.

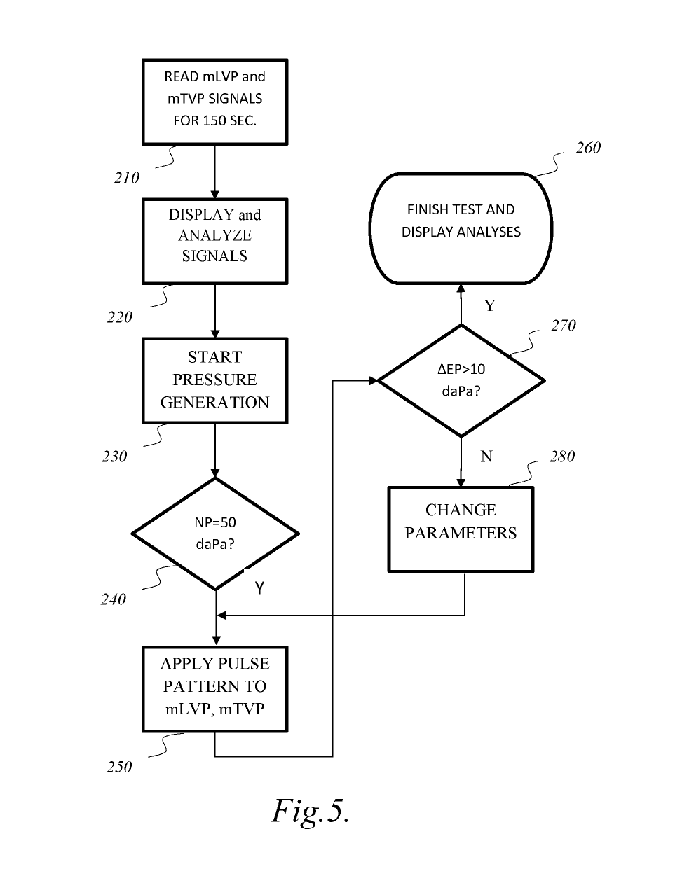

FIG. 6 shows the shape of the stimulation pulses applied to mLVP and mTVP muscles. In FIG. 6, axis 480 shows time in Seconds and axis 490 shows signal amplitude in Volts. Item 440 is mTVP pulse which starts at 450, lasts for 0.020 seconds and finishes at 470. The amplitude of the signal 420 is 0.2 mV. If mTVP (440) signal fails to open ET, the process is repeated with mLVP (430), then with mTVP (440) and mLVP (430) together. When both mLVP and mTVP stimulated together, starting time 435 of mLVP should be before the starting time 450 of mTVP. In the FIG. 460 is the finishing time of mLVP signal. 410 and 420 show signal amplitude of mLVP and mTVP signals.

The ETD assessment of the patient is based on the results of the aforementioned test results.
The following are the assessment categories:
Type 0: ET does not open under no circumstances after all test patterns,
Type 1: ET opens without external stimulation
Type 2: ET opens with only mTVP stimulation with minimal pulse parameters,
Type 3: ET opens with both mLVP and mTVP stimulations with minimal pulse parameters,
Type 4: ET opens with both mLVP and mTVP stimulations with increased pulse parameters.

Medical interpretations of these types are beyond the scope of this document but they broadly define the ETD assessment level.

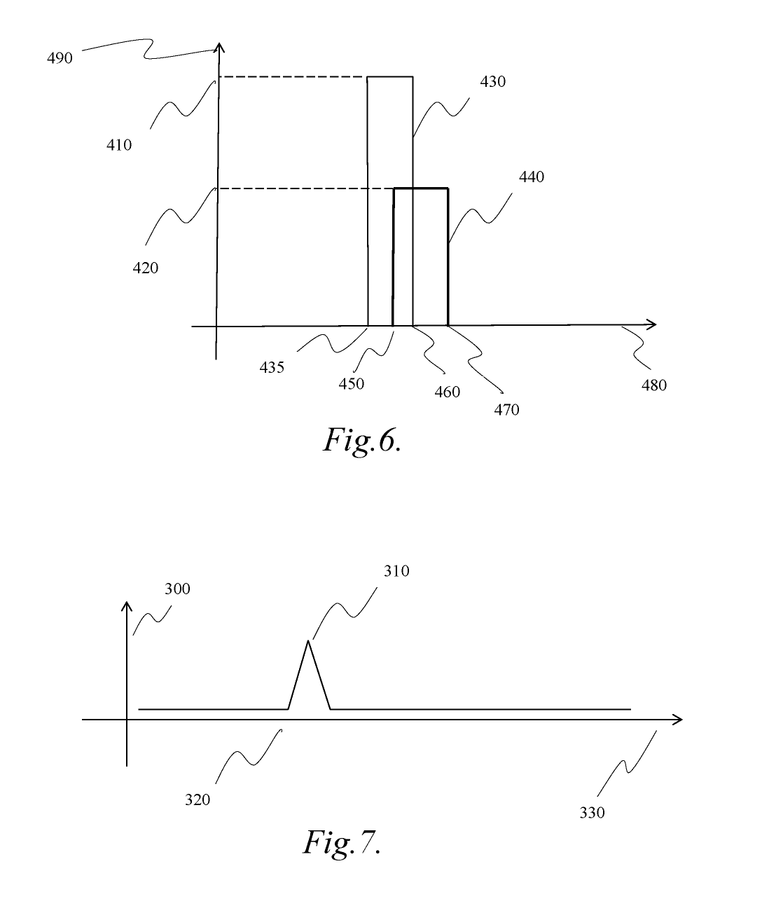

FIG. 7 shows the type of pressure change expected in external ear canal sensor output during opening of ET. Vertical axis 300 shows the pressure in daPa range, while horizontal axis 330 shows time scale. Item 310 shows the instant of ET opening at time indicated by 320. Normally 320 is the time of application of the stimulus when pressure change (AP) of at least 10 daPa is observed.

1. A system for diagnosis of Eustachian Tube Dysfunction ailment, the system comprising:
a probe assembly adapted to connect to one or more Eustachian tube related muscles locally over skin inside palatal area,
a set of one or more sensors to detect opening of Eustachian tube,
a data acquisition unit with one or more signal conditioners to condition incoming and outgoing signals,
a processing computer in communication with the data acquisition unit wherein the processing computer executes a software to control operation of the system,
a set of one or more sensors to measure nasal pressure; and
a pressure generator to increase pressure inside nasal cavity.

2. The system according to claim 1 wherein the probe assembly adapted to connect to one or more Eustachian tube related muscles locally over skin inside palatal area comprises one or more electrodes selected from a subdermal electrode, a surface electrode, an intramuscular electrode, an intraneural electrode, an optical electrode, an optical probe, or a combination of the foregoing electrodes and probes.

3. The system according to claim 1 wherein the set of one or more sensors to detect opening of Eustachian tube comprises one or more sensors selected from a pressure sensor, a distance sensor, or a combination of the foregoing sensors placed on external ear canal of patient.

4. The system according to claim 1 wherein the data acquisition unit with one or more signal conditioners to condition incoming and outgoing signals comprises one or more subsystems to generate outgoing electrical pulses, optical pulses and one or more subsystems to condition incoming electrical pulses, optical pulses or a combination of the foregoing subsystems.

5. The system according to claim 1 wherein the set of one or more sensors to measure nasal pressure comprises one or more pressure sensors selected to be placed nasally to nose, orally to mouth, on a mask covering mouth or a combination of aforementioned locations.

6. A method for diagnosing Eustachian Tube Dysfunction problem by utilizing Eustachian tube muscles comprising a Tensor Veli Palatini muscle, a Levator Veli Palatini muscle and a Tympanic membrane, the method comprising:
increasing pressure inside nasal cavity,
applying stimulation pulses to one or more Eustachian tube muscles using a probe assembly,
monitoring sign of movement of Tympanic membrane; and
in case no movement of the Tympanic membrane is observed, repeating the foregoing steps after changing stimulation pulse parameters of the Tensor Veli Palatini and the Levator Veli Palatini muscles.

7. The method set forth in claim 6, wherein increasing pressure inside the nasal cavity is achieved externally by external pressure generator.

8. The method set forth in claim 6, wherein increasing pressure inside the nasal cavity is achieved by patient performing Valsalva maneuver.

9. The method set forth in claim 6, wherein the stimulation pulses are applied to one or more Eustachian tube muscles using the probe assembly electrically.

10. The method set forth in claim 6, wherein the stimulation pulses are applied to one or more Eustachian tube muscles using the probe assembly optically.

11. The method set forth in claim 6, wherein monitoring sign of movement of Tympanic membrane is done by external ear canal sensors.

12. The method set forth in claim 6 wherein in case no movement of the Tympanic membrane is observed, repeating the foregoing steps after increasing amplitude of the stimulation parameters to Tensor Veli Palatini muscle, Levator Veli Palatini muscle or both.

13. The method set forth in claim 6 wherein in case no movement of the Tympanic membrane is observed, repeating the foregoing steps after increasing duration of the stimulation parameters to Tensor Veli Palatini muscle, Levator Veli Palatini muscle or both.

14. The method set forth in claim 6 wherein in case no movement of the Tympanic membrane is observed, repeating the foregoing steps after increasing the difference between starting time of the stimulation to Tensor Veli Palatini muscle and the Levator Veli Palatini muscle.
